(19) United States
(12) Patent Application Publication
(10) Pub. No.: US 2025/0275847 A1
(43) Pub. Date: Sep. 4, 2025

(54) Spiral Flow-Inducing Exo-Graft

(71) Applicant: Drexel University, Philladelphia, PA (US)

(72) Inventors: J Yasha Kresh, Yardley, PA (US); Pablo Huang Zhang, Philadelphia, PA (US); Colin Tkatch, Philadelphia, PA (US)

(21) Appl. No.: 18/214,880

(22) Filed: Jun. 27, 2023

Related U.S. Application Data
(63) Continuation of application No. 16/924,247, filed on Jul. 9, 2020, now abandoned.
(60) Provisional application No. 62/871,829, filed on Jul. 9, 2019.

(51) Int. Cl. A61F 2/06; A61B 17/11; A61M 60/148; A61M 60/161; A61M 60/289; A61M 60/857

(52) U.S. Cl. CPC A61F 2/06; A61M 60/148; A61M 60/161; A61M 60/289; A61M 60/857; A61B 2017/1135; A61M 2206/16

(57) ABSTRACT

A spiral flow-inducing exo-graft is a non-blood contacting helically shaped device that wraps around the outside of a blood-carrying conduit and manipulates hemodynamic flow-patterns. The blood-carrying conduit can be a natural tissue blood vessel or an artificial graft.

### CROSS-REFERENCE TO RELATED APPLICATION

The present application is a continuation of U.S. patent application Ser. No. 16/924,247, which claims the benefit of U.S. Provisional Patent Application Ser. No. 62/871,829, filed on Jul. 9, 2019, both of which are incorporated herein by reference in their entireties.

### BACKGROUND OF THE INVENTION

Heart disease is the leading cause of death in the United States, annually contributing to about a quarter of all deaths nationwide. Technological advancements of continuous-flow mechanical circulatory support (MCS) devices for patients with heart failure have increasingly led to their use not only as a bridge-to-transplant, but as a destination therapy. Although current MCS devices can maintain blood circulation, complications such as bleeding, thromboembolic events, infection, and inflow suction-induced arrhythmias limit optimal outcomes. Using biologically inspired spiral forms of flow, this work explores how the manipulation of MCS outflow characteristics can ameliorate device-mediated vascular complications.

Heart failure occurs when the heart is unable to pump enough blood to meet the body's needs. This is a chronic and progressive condition in which the heart cannot maintain needed demand. Among the many treatment alternatives/options available (medical-Rx, surgical), MCS is the only available large-scale device (heart-transplantation is limited to <2,000 cases/year) for replacing the pump function of a weak or failing heart. MCS devices function by pumping blood into the aorta from the left- and/or right-ventricle (to pulmonary artery) via a dedicated cannula. The heart failure population in the US alone is approaching 5.7 million people of which >150,000 patients are estimated to be candidates for receiving this technology annually.

The US and global mechanical circulatory support market is expected to increase at significant growth rates and is supported by various growth drivers, such as, long waiting list for heart transplant treatment, launches of new products, improved results of MCS relative to other treatment options. Despite their mechanical/hemodynamic improvements however, these devices continue to generate non-physiological straight-flow patterns leading to complications listed above.

It would be beneficial to provide mechanical circulatory support by means and devices that induce a more natural spiral flow.

### BRIEF SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Spiral/helical forms of flow are expected to be more vascular-form conforming (i.e., out-of-plane aortic arch) than straight flow and thereby provide crucial hemodynamic benefits. The present invention demonstrates that spiral flow is superior in number of ways. It is shown to reduce the detrimental regions of flow instability, low velocity, and low wall shear stress (WSS), key determinants of vascular biology/pathology.

Recapitulating spiral blood flow in mechanically assisted circulation will help minimize/eliminate several device-related adverse outcomes. Spiral fluid modulation as an added parameter and design consideration in the use of conventional MCS devices is expected to be more adaptable to patient-specific vascular system, reducing/eliminating risks of athero-thrombosis.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a model of a patient-specific aorta;

FIG. 1A is a schematic drawing of an outflow graft of an aorta virtually anastomosed end-to-side to the ascending aorta in a lateral position;

FIG. 1B is a schematic drawing of the anastomosed graft directed inferiorly/superiorly by 20°;

FIG. 1C is a schematic drawing of the anastomosed graft directed anteriorly/posteriorly by 20°;

FIG. 2 is a computationally simulated velocity streamline representation of baseline unaltered flow in an aorta;

FIG. 2A shows computationally simulated velocity streamlines resulting from clockwise/counterclockwise flow directions using rotational speeds of 70 RPM, 160 RPM, 318 RPM, and 500 RPM as outflow graft input condition;

FIG. 3A is a bar chart showing the volume of regions having low velocity for the baseline lateral graft position shown in FIG. 1A;

FIG. 3B is a bar chart showing areas of low wall shear stress (WSS) for the baseline lateral graft position shown in FIG. 1A;

FIG. 3C is a bar chart showing areas of high WSS for the lateral graft position shown in FIG. 1A;

FIG. 4 shows computationally simulated velocity streamlines and super-imposed low-WSS regions resulting from superiorly and anteriorly directed outflow graft configurations, compared to baseline lateral graft position, shown in FIG. 1B;

FIG. 5A is a bar chart showing the volume of regions having low velocity resulting from superiorly and inferiorly directed outflow graft positions shown in FIG. 1B;

FIG. 5B is a bar chart showing areas of low WSS resulting from superiorly and inferiorly directed outflow graft positions shown in FIG. 1B;

FIG. 5C is a bar chart showing areas of high WSS resulting from superiorly and inferiorly directed outflow graft positions shown in FIG. 1B;

FIG. 6 is a top view of the computationally simulated velocity streamlines and added straight-flow trajectory resulting from anteriorly and posteriorly directed outflow graft positioning, with respect to the baseline lateral position, shown in FIG. 1C;

FIG. 6A is a bar chart showing the volume of regions having low velocity resulting from anteriorly and posteriorly directed outflow graft positions shown in FIG. 1C;

FIG. 6B is a bar chart showing areas of low WSS resulting from anteriorly and posteriorly directed outflow graft positions shown in FIG. 1C;

FIG. 6C is a bar chart showing areas of high WSS resulting from anteriorly and posteriorly directed outflow graft positions shown in FIG. 1C;

FIG. 7 is an exemplary embodiment of the present invention and schematic view of the exo-graft enveloping the outflow graft of the mechanical circulatory support device;

FIG. 7A is a front elevational view of an exo-graft according to a first exemplary embodiment of the present invention;

FIG. 7B is a side elevational view of the exo-graft of FIG. 7A;

FIG. 8 is an additional exemplary embodiment of the present invention and schematic view of the exo-graft enveloping the outflow graft of the mechanical circulatory support device;

FIG. 8A is a front elevational view of an exo-graft according to a second exemplary embodiment of the present invention;

FIG. 8B is a side elevational view of the exo-graft of FIG. 8A;

FIG. 9 is a perspective view of an outflow graft according to an exemplary embodiment of the present invention;

FIG. 10 is a side elevational view of the outflow graft of FIG. 9;

FIG. 11 is a perspective view of an outflow graft according to another exemplary embodiment of the present invention;

FIG. 12 is a side elevational view of the outflow graft of FIG. 11;

FIG. 13 a side elevational view of a ribbed outflow graft according to an exemplary embodiment of the present invention;

FIG. 14 is a side elevational view of an accordion outflow graft according to an exemplary embodiment of the present invention; and

FIG. 15 is a side elevational view of a a slotted exo-graft according to an exemplary embodiment of the present invention.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range. The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

Spiral/helical forms of blood flow have been observed in many locations in the cardiovascular system. Mechanical circulatory support (MCS) devices, despite their increased use in end-stage heart failure, generate non-physiological flow regimes. Computational fluid dynamic studies and benchtop flow experiments have demonstrated objective benefits for inducing spiral forms of flow in the MCS outflow graft. Recapitulating spiral flow reduces the impact jet pressure at the outlet, normalizes wall shear stress gradients, and reduces the size of flow stasis/recirculation zones, creating highly desirable conditions that minimize/eliminate adverse outcomes.

Spiral blood flow, consisting of combined axial and circumferential flow fields, has been identified throughout the cardiovascular system and postulated to offer several hemodynamic benefits, including the normalization of wall shear stress (WSS) gradients. WSS is a fluid-velocity-induced tangential force that plays an important role in endothelial cell mechano-signaling. It is well understood that prolonged exposure to low WSS induces flow-mediated endothelial cell misalignment, leading to increased vascular wall permeability and subsequent atherothrombotic risk/development.” Conversely, excessively high WSS can lead to vascular wall fatigue and platelet activation.

Although several studies have looked at the outflow graft positioning and angle, few have evaluated the aortic hemodynamics of incorporating spiral flow mechanics to the MCS outflow. In the presented research we aimed to study the biomechanical and hemodynamic impact of spiral forms of flow at the MCS outflow graft-aorta interface. It is well known that the fluid jetting caused by MCS devices drastically alters the aortic flow patterns, introducing pathophysiologic flow regimes affecting the vascular response. The effects of the intensity of spiral flow modulation and the insertion angle of the outflow graft were evaluated in terms of previously established benefits: normalization of WSS gradients, diminishing high-velocity jet-flow on the vascular wall (impingement), and reduction of regions of low flow. This work is not only expected to inform future MCS device designs by elucidating the beneficial role of spiral flow as an organizational force, but also to provide greater understanding in surgical practice for minimizing progression of flow-induced vascular disease and related adverse clinical outcomes (eg, intimal hyperplasia, atheroma erosion).

### Material and Methods

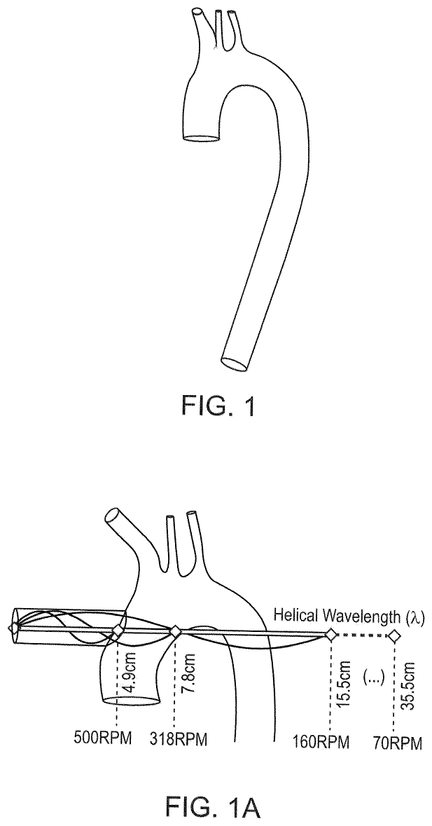

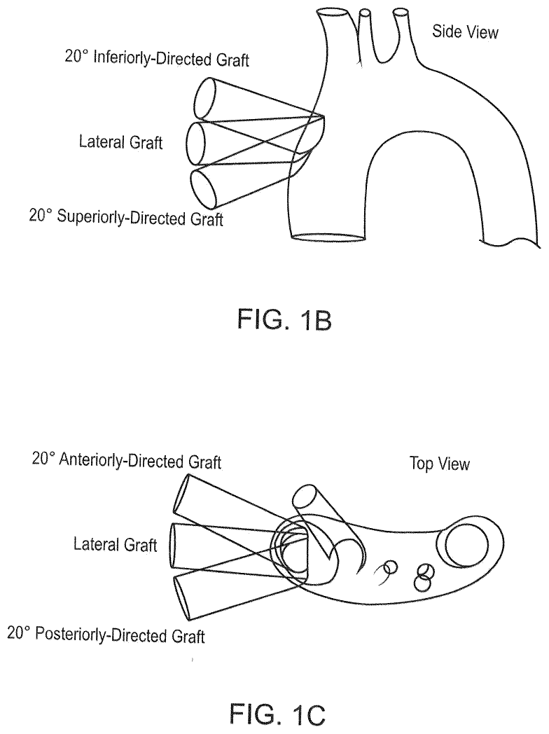

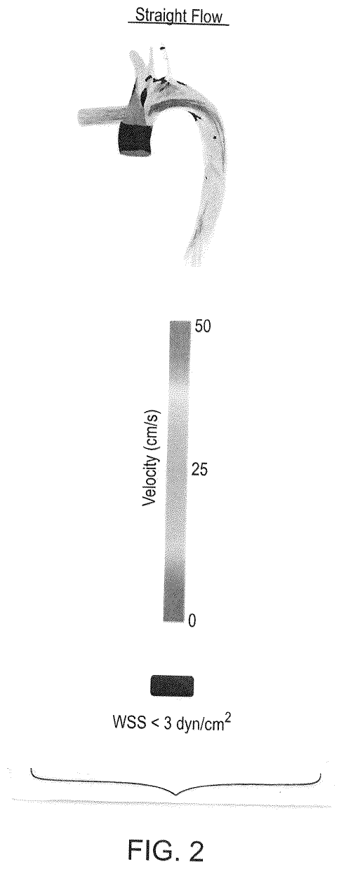

Three computational fluid dynamic (CFD) sub studies were conducted using a three-dimensional (3D)-image-derived patient aortic arch model (FIG. 1) obtained from an online repository (Stratasys, Cambridge, MA). This model was modified using SolidWorks (Dassault Systemes, Velizy-Villacoublay, France) to include a laterally positioned outflow graft (1.6 cm diameter) virtually anastomosed end-to-side 2 cm below the brachiocephalic trunk (FIG. 1A). The first sub-study evaluated the effects of spiral flow modulation, introduced at the laterally positioned outflow graft, on aortic arch hemodynamics. To define the spiral flow intensity, the rotation (revolutions per minute [RPM]) and direction (+/− denoting clockwise/counterclockwise, respectively) of the graft outflow was set to 0, ±70, ±160, ±318, and ±500 RPM. Modeling a patient-specific aorta, shown in FIG. 1, and using straight flow as control (FIGS. 2) and ±160 RPM for the spiral flow conditions, the second and third sub-studies evaluated the combined effects of the graft insertion angle and spiral flow content. With respect to the laterally positioned graft anastomosis (serving as the zero-reference position), the graft was directed inferiorly/superiorly by 20° (second sub-study), shown in FIG. 1B, and directed anteriorly/posteriorly by 20° (third sub-study), shown in FIG. 1C.

### Computational Fluid Dynamic Simulations

The open source software OpenFOAM (ESI, Bracknell, UK) was used for the 3D mesh generation and fluid dynamic simulations. The mesh for the aorta model consisted of 1.5 million hexahedral cells. A mesh refinement study was completed using the outflow graft fluid jet velocity and the impact WSS as convergence parameters to ensure that the computed results were representative of the flow dynamics (given the geometry and initial conditions) and not due to insufficient analytical-grid resolution of the fluid domain. The working fluid (blood) was modeled as an incompressible Newtonian fluid with a density of 1.06 g/cm³ and a viscosity of 4.1 cP. A transient solver incorporating turbulence modeling was used to solve the equations (Navier-Stokes) governing fluid dynamics.

No-slip boundary conditions were implemented at all vessel walls, which were assumed to be rigid. Continuous flow 5 L/min was generated at the graft simulating MCS outflow, and 1 L/min at the aortic root inlet simulating the impaired cardiac output. Spiral flow with variable helical content was implemented at the graft inlet using a rate of rotation (RPM) condition. The 70 RPM was chosen to mimic the average human heart rate of 70 beats per minute; 318 RPM was used to generate a single helix with a wavelength equivalent to the distance between the modeled graft inlet and the inner curvature of the ascending aorta; 160 RPM generated a wavelength twice that of 318 RPM; 500 RPM generated a wavelength equal to the graft length used in the model (FIG. 1A). Straight (0 RPM) flow condition was used at the aortic root inlet boundary and also served as the control case for the MCS outflow graft.

### Analysis and Visualization

Post-processing/analysis was conducted using an open-source visualization software (Para View, Kitware, NY). To evaluate flow disturbance and high-velocity fluid jetting, velocity-colored streamlines and areas of WSS were visualized. To quantify and assess the size of atheroprone regions and the impact of high fluid velocity jets, volumes of low velocity (<5 cm/s), areas of low WSS (<3 dyn/cm²), and areas of high WSS (>80 dyn/cm²) were extracted using threshold analysis methods. The low-WSS cutoff value is associated with atherothrombotic risk due to flow-mediated endothelial dysfunction, and the high-WSS value is associated with shear-induced platelet activation.

### Results

### First Sub-study: Laterally Positioned Outflow Graft Spiral Flow Modulation

The velocity-colored streamlines resulting from the spiral flow modulation at the inlet of the end-to-side, laterally placed outflow graft are shown in FIGS. 2 and 2A. Areas of low WSS were superimposed on the streamlines to identify regions vulnerable to endothelial dysfunction. Despite the induced straight flow at the graft, the control case exhibited swirling features at the descending aorta, attributed primarily to the additive effects of the aortic arch curvature and 3D torsional geometry of the ascending and descending segments (out-of-plane geometry). The “braiding/weaving” of the streamlines was significantly different for the straight, clockwise, and counterclockwise flow conditions. As the helical-flow content increased, the high-velocity jetting (red streamlines, FIGS. 2 and 2A) at the arch diminished, affecting the size of the recirculation pockets at the descending segment of the aorta. Concurrently, the areas of low WSS at the aortic root decreased in size with increasing helical-flow content.

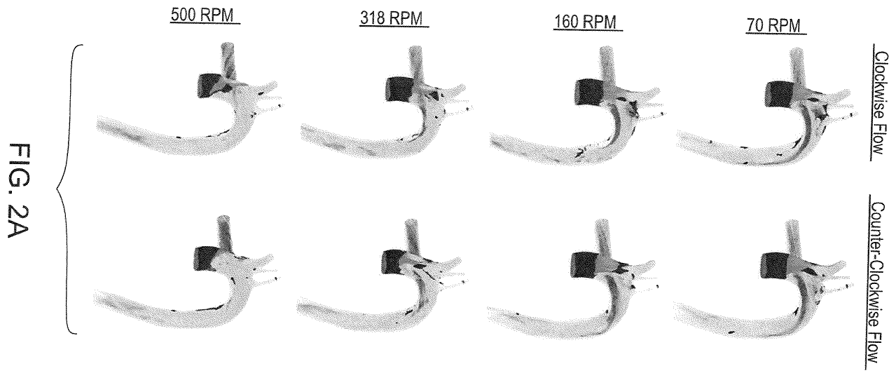

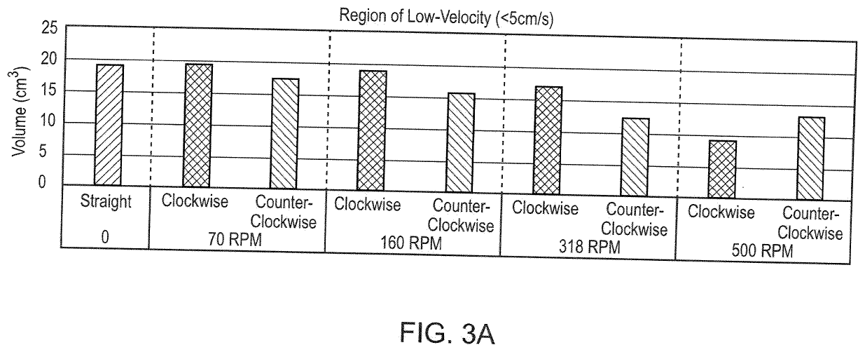

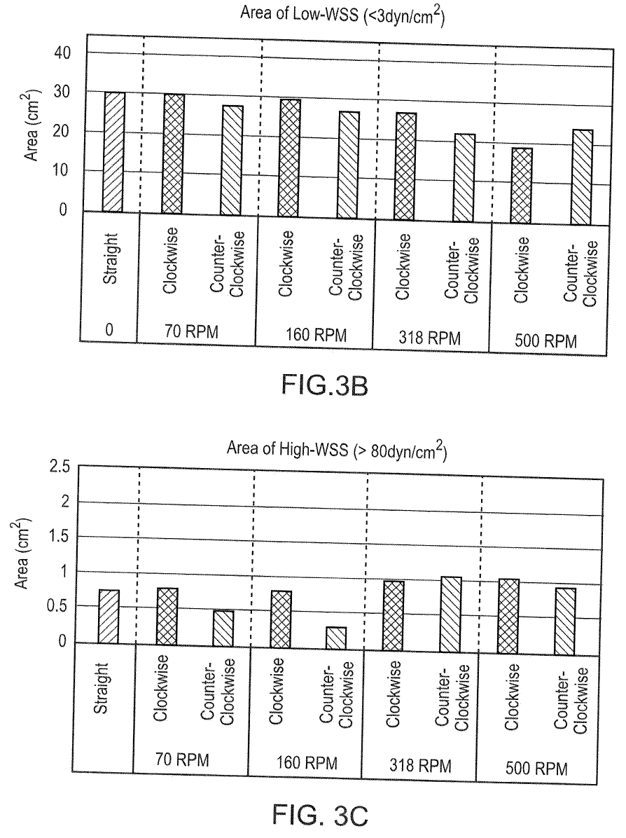

Quantitatively, as the helical-flow content increased, the volume of low velocity in the fluid domain diminished (FIG. 3A). With the exception of the 500 RPM case, counterclockwise graft flow predominantly minimized regions of low velocity. With the 500 RPM case, clockwise flow decreased the volume of low velocity by 2.1-fold and counterclockwise by 1.4-fold, when compared with straight flow.

Spiral flow markedly altered the WSS response, principally decreasing the size of low WSS area with increasing helical-flow content (FIG. 3B). At the highest helical-flow content, clockwise flow exhibited a 1.5-fold decrease and counterclockwise flow yielded a 1.2-fold reduction in low-WSS area when compared with straight flow (FIG. 3B). Despite the reduction of low-WSS areas, higher helical-flow content (eg, 500 RPM) demonstrated increased size of high-WSS areas at the ascending aorta. The highest helical-flow content had on average a 1.3-fold size increase in high WSS area (actual values exceeding 150 dyn/cm²) when compared with straight flow (FIG. 3C). The 160 RPM counterclockwise flow regime exhibited the smallest area of high WSS, while also reducing the size of regions with low velocity and areas of low-WSS. This spiral flow regime was used for 2 subsequent outflow graft insertion angle variation sub-studies.

### Second Sub-study: Inferiorly/Superiorly Directed Outflow Graft

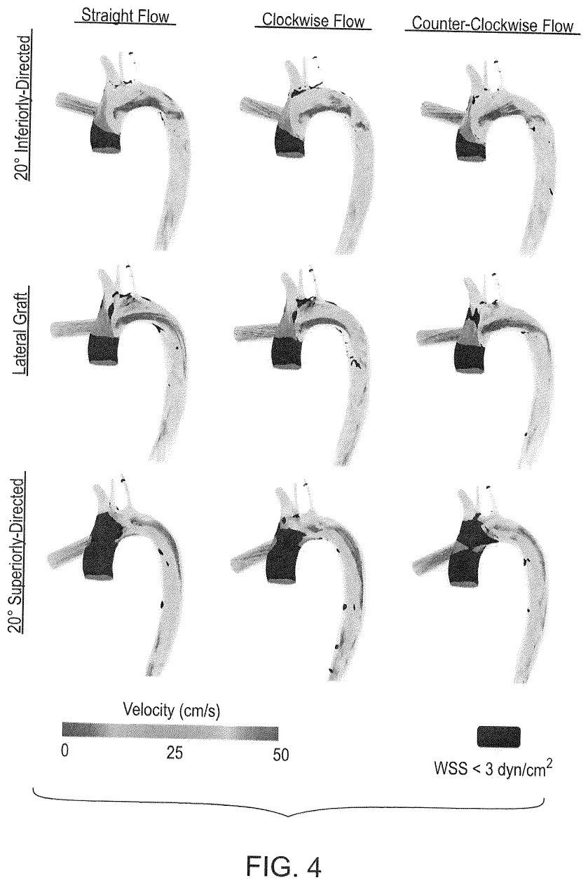

To study the combined effects of MCS spiral outflow and graft angle variation on aortic hemodynamics, the graft anastomosis angle was directed inferiorly and superiorly (by 20°) with respect to the lateral positioning. The angle of the graft affected the distribution of the high velocities (colored in red) at the arch and the low-WSS areas at the aortic root (FIG. 4). The inferiorly directed graft had the best clearance of the areas of low-WSS at the root, but caused the high-velocity jets to concentrate on the inner curvature of the ascending aorta, creating an impingement force on the wall. The superiorly directed graft focused high velocities towards the distal wall of the descending aorta, but resulted in the largest areas of low WSS at the aortic root.

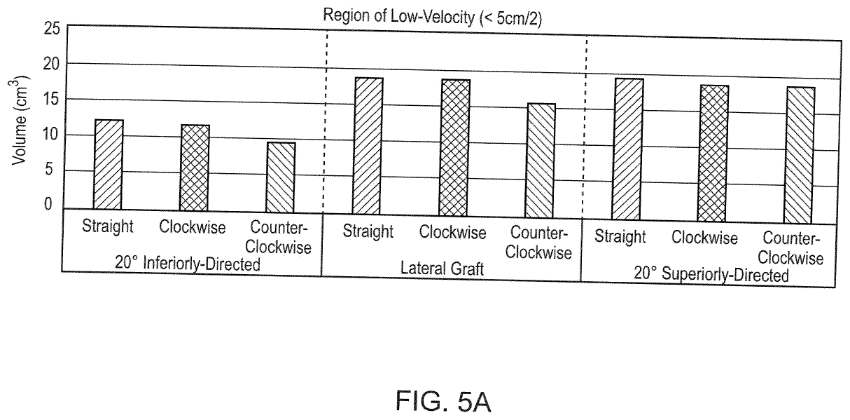

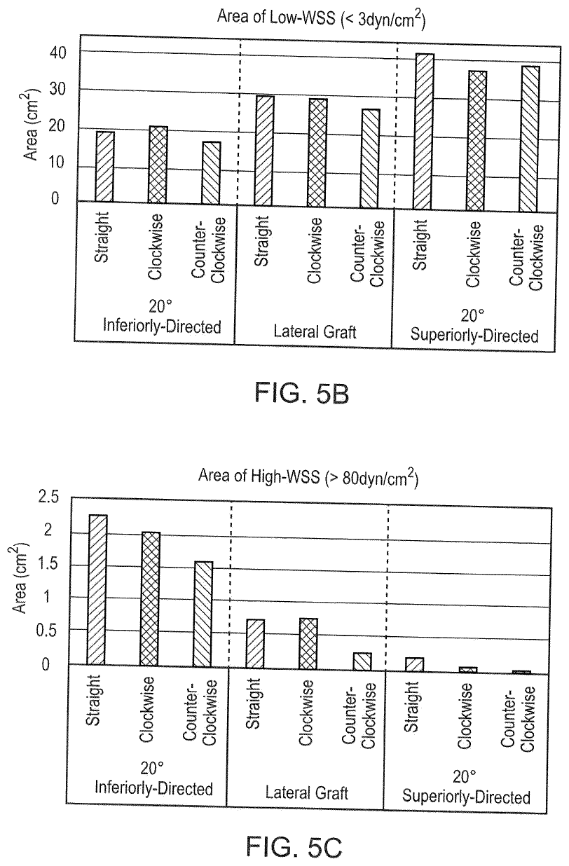

The inferiorly directed graft anastomosis demonstrated smaller volumes of low velocity and diminished areas of low WSS. Straight flow regime for the inferiorly directed graft had on average a 1.6-fold reduction in low velocity volume compared with other graft angles (FIG. 5A). Counterclockwise graft flow improved predominantly the clearance of low velocity and low-WSS (FIG. 5B), and decreased areas of high-WSS (FIG. 5C) created by the fluid-jet impingement, compared with straight and clockwise flows. For the inferiorly directed and lateral grafts, there was a 1.2-fold decrease in low velocity for counterclockwise flow compared with straight flow; superiorly directed graft positioning had a marginal reduction in low velocity with counterclockwise flow, compared with straight flow. Although the inferiorly directed graft positioning was better in diminishing low-WSS areas, it increased the areas of high-WSS, specifically at the fluid jet impact site located contralaterally to the graft outflow (ie, inner curvature of the arch). However, when compared to straight flow, inducing a clockwise flow in the inferiorly directed outflow graft yielded a 1.1-fold decrease in size of high-WSS areas, while counterclockwise flow yielded a 1.4-fold decrease.

### Third Sub-study: Anteriorly/Posteriorly Directed Outflow Graft

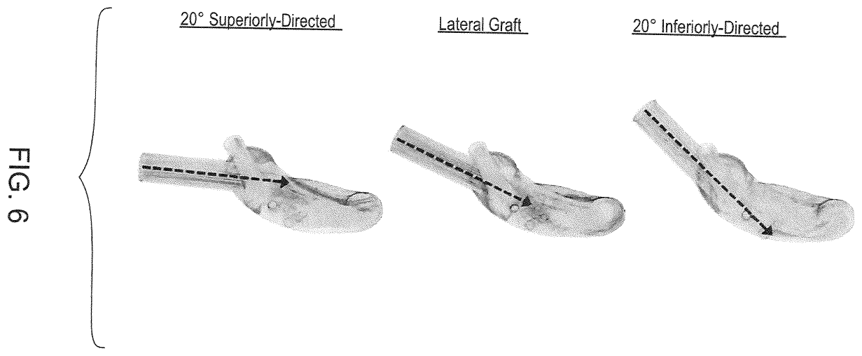

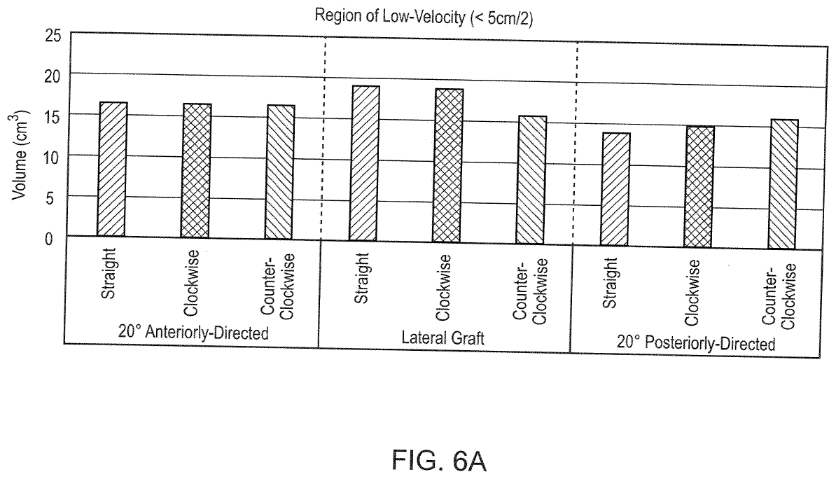

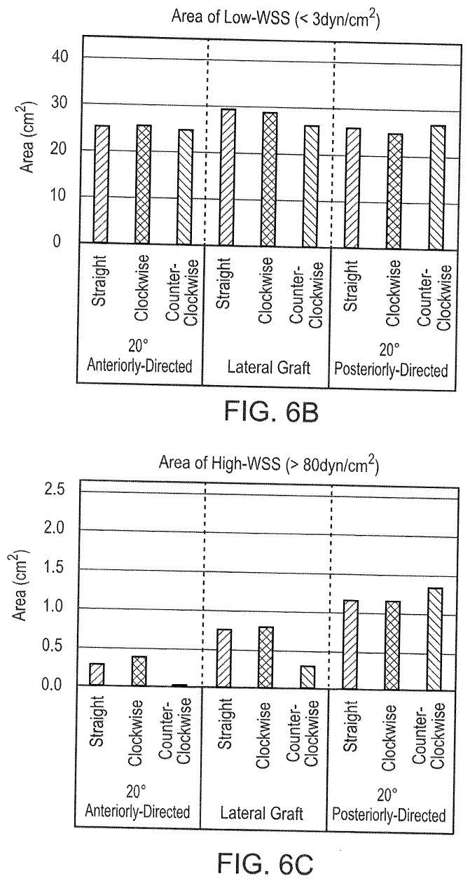

The anastomosis angle of the MCS outflow was additionally varied in the transverse plane, directing the graft anteriorly and posteriorly by 20° with respect to the lateral-positioning, as shown in FIG. 1C. In the posteriorly directed graft, the induced spiral flow increased the size of low-velocity regions (FIG. 6A) and areas of low-WSS (FIG. 6B). In the anteriorly directed anastomosis there was no significant improvement in these values with imposed spiral flow. However, high-WSS (FIG. 6C) was significantly affected by varying the transverse angle and adding spiral flow. The anteriorly directed graft demonstrated a substantially smaller area of high WSS, where counterclockwise flow produced a 13.2-fold reduction compared with straight flow. For the posteriorly directed graft, counterclockwise flow yielded a 1.15-fold increase in high-WSS area size compared with straight flow. This distinct difference when using counterclockwise flow can be attributed to the resultant anastomosis-angle-induced fluid jet vascular wall impingement.

The goal of this study was to create an in silico platform to formulate an integrative understanding of spiral blood flow generated at the MCS outflow graft and the resultant aortic hemodynamics. The prevailing 3D curvature of the aorta imparts a natural spiral motion on blood flow that is unique for each patient. These aortic hemodynamic signatures are significantly altered by current MCS devices, which not only extinguish the native spiral flow patterns but also introduce disruptive fluid jets, causing recirculation zones and pathogenic regions of low WSS, increasing the risk of vascular disease progression and complications (eg, intimal hyperplasia, atheroma disruption/erosion). The hemodynamic outcomes of introducing spiral flow through an outflow graft, virtually anastomosed end-to-side to the ascending aortic arch of a patient-specific aorta model, were studied in detail.

The first sub-study demonstrated that by increasing the helical-flow content, the high velocities at the arch were diminished and the size of flow recirculation/stasis regions were reduced. The blood transport benefits of spiral flow were similarly observed by others. Despite the improvement in areas of low WSS, increasing helical-flow content (ie, 500 RPM) also increased the areas of WSS in excess of 80 dyn/cm², with overall highest measured peak WSS ˜150 dyn/cm². This sub study provided an important insight on spiral flow generation, establishing that a defined spiral structure (set helical intensity/direction) is not generic, requiring that the 3D vascular geometry of the individual patient be considered.

In the second sub-study, the inferiorly directed graft positioning demonstrated the smallest regions of low-velocity and reduced areas of low-WSS, due to the better-aimed washout of the aortic root. However, this configuration also increased the impact area WSS greater than 80 dyn/cm² at the ascending aorta, which was moderated by the imposed spiral flow regimes (ie, counter-clockwise). Whereas counterclockwise spiral flow improved overall washout parameters for the inferiorly directed graft, the superiorly directed graft fared marginally better with clockwise flow. The difference was largely due to the arch configuration itself; flow from the graft in the superiorly directed position did not need to navigate the aortic curvature, aiming directly towards the descending aorta. Accordingly, the impact WSS areas were the smallest for the superiorly directed graft.

The third sub-study emphasized the impact of transverse graft positioning on high-WSS areas. As illustrated in FIGS. 6-6C, the transverse anastomosis placement significantly alters the expected flow trajectory, and consequential mechanical impingement on the wall. The anteriorly directed graft had a considerably diminished wall impact (impingement) than the laterally placed graft. The marked decrease in high WSS in response to the counterclockwise flow in the anteriorly directed graft is a reflection of the inherent helical continuity of the fluid structure. This is a striking example of “form follows function”, where the modeled 3D aortic arch geometry naturally induced a counterclockwise spiral flow direction. Clockwise flow in the anteriorly directed graft increased the wall impact, necessitated by the rotational switchback (from clockwise to counterclockwise) dictated by the aortic 3D curvature. Conversely, the immediacy of the flow impingement on the posterior wall in the posteriorly directed graft generated increased areas of high WSS. Given the posterior location of the fluid impact, counterclockwise graft flow caused the fluid to be forced into the wall, increasing the high WSS. In this specific case, increasing the helical-flow intensity (higher RPM) in the clockwise direction may help circumnavigate the wall and thus minimize the fluid impact-force.

In evaluating the resultant complex aortic flow dynamics, spiral flow generation was shown to be instrumental in addressing problem regions due to outflow graft fluid jetting. The study provides several translational considerations pertinent to MCS surgical implant approaches. Orienting the MCS outflow graft to the downstream flow is preferable for minimizing areas of high WSS (associated with vessel wall fatigue/weakening, erosion of pre-existing atheroma, and subsequent embolic complication). With limited surgical window and accessibility, the graft placement should be directed to avoid nearby fluid jet impingement on the vascular wall. Spiral flow is expected to have broad utility: Low helical-flow content can be used to navigate downstream vessel tortuosity in cases where the anastomosis positioning is unrestricted, and high helical-flow content can be used when access is limited and collision of the fluid jet with the vessel wall is unavoidable. Despite the benefits of spiral flow, the jet impact location (anterior/posterior or proximal/distal to the arch) and the preferred fluid-directing rotation of the native aorta should be carefully considered to avoid “switchback” in spiral directionality, which may contribute to the formation of disturbed flow. Spiral flow must be optimized and informed by the native geometry and flow/anastomosis conditions to prevent detrimental flow form and vascular-structure mismatching.

There are a number of limitations to numerical modeling in general that need to be acknowledged. In particular, the aortic walls were modeled as rigid boundaries to simplify the chosen computational approach. Although these studies focused on continuous flow with added spirality, it is important to recognize that in mechanically supported circulation the composite aortic flow preserves some pulsatility. Considering that spiral flow was shown to reduce high-velocity jetting, its role in ameliorating aortic dysfunction (eg, insufficiency) shows promise and requires further investigation. Importantly, this analysis was confined to a single aorta model template, and although it did not address the full range of anatomical variations or exhaustively test all the degrees of freedom of the graft anastomosis positions, it created a working framework for testing explicit angles and conditions pertaining to individual surgical cases.

In conclusion, using an in silico approach, a framework for understanding spiral flow and its associated benefits in assisted circulation was implemented in a patient-derived model of the aortic arch with a virtually anastomosed MCS-pump outflow graft. Specifically, the impact of spiral modulation of the graft outflow and the effect of the graft angle positioning in relation to the ascending aorta were studied. The results demonstrated that increased spiral flow reduced regions of low-velocity and areas of low-WSS, important hemodynamic determinants of vascular endothelial dysfunction. When optimized, matching of helical-flow content to the patient-specific vascular anatomy and flow conditions (rotational direction, helical pitch), spiral flow can be used to minimize endothelial-disruption by fluid jetting and improve washout of recirculation/stasis problem zones. MCS device designs incorporating spiral-flow generating features may prove to be a desirable solution improving the hemodynamics of the aorta-outflow graft coupling. Several implantable medical devices (eg, endovascular grafts, valves, cardiopulmonary bypass cannula) also stand to benefit from spiral flow-induced reduction of fluid flow disturbances and related mechano-biological molecular signaling responses, improving patient-individualized therapy.

A Spiral Flow-Inducing Exo-Graft (i.e., Spiral Exo-Guide) according to an exemplary embodiment of the present invention, is a non-blood-contacting helically-shaped device that will serve as an add-on to existing commercial MCS devices. The exo-graft is designed to act as a sleeve wrapping around the outside of the outflow graft distal to the pump and is intended to manipulate the pump-generated flow. Modulating the helical-flow content (e.g., helical pitch/revolutions, left/right-handed helix) is expected to minimize fluid jetting, vascular thrombosis and endothelial cell dysfunction (e.g., stroke, bleeding).

MCS devices stand to benefit from this add-on device that does not require modifying the underlying pump design. Generation of spiral flow and its benefits are also applicable to extracorporeal circulation cannulas, vascular grafts, and hemodialysis access ports. The exo-graft can also be used as a native blood flow-modulator enveloping large native blood vessels (eg, aorta/brachiocephalic/carotid/iliac/femoral sleeve).

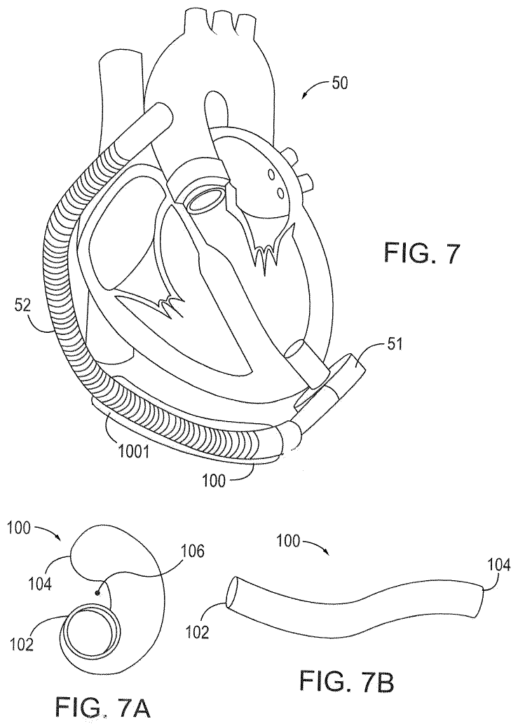

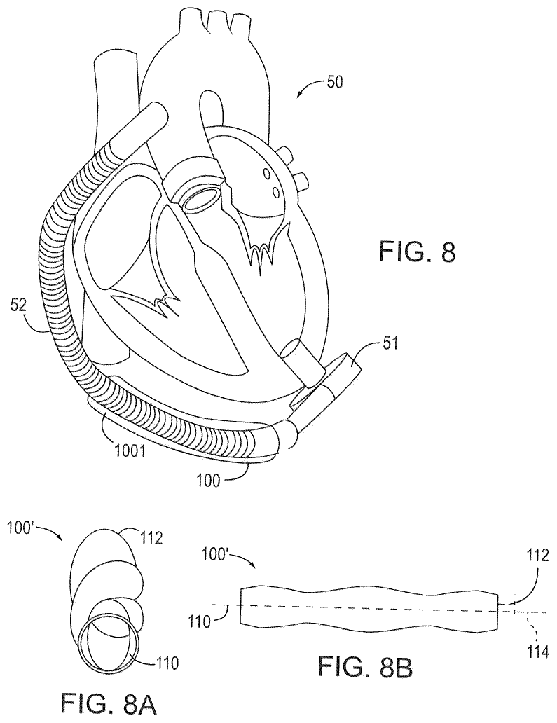

An exemplary embodiment of the present invention is shown in FIGS. 7-8B. FIGS. 7 and 8 each shows a heart 50 with a MCS system having a pump 51 and an outflow graft 52 that is part of the MCS system. An exo-graft 100, 100′ according to two alternative exemplary embodiments of the present invention can be applied over the exterior of the outflow graft 52, downstream of pump 51, as shown in FIGS. 7 and 8.

The exterior shell of exo-grafts 100, 100′ is provided so as to impart and control spiral flow structures within the outflow graft 52 itself. The design lends itself to modulating helical flow content (e.g. pitch, revolutions, left/right-handed helix), enabling effective programmability and patient-specific matching. Using biologically-inspired spiral forms of flow, the exo-graft 100, 100′ is designed and intended to manipulate the hemodynamics of the graft flow and ameliorate device-mediated complications such as jetting, vascular remodeling, thrombosis, and downstream endothelial dysfunction.

Referring to FIGS. 7A and 7B, an exemplary exo-graft 100 has a generally arcuate or helical profile such that a cross section of a lower part 102 of exo-graft 100 does not intersect with a cross section of an upper part 104 of exo-graft 100 along a line parallel to a central longitudinal axis 106 (extending perpendicularly from the plane of the page of FIG. 7A) of exo-graft 100.

Referring to FIGS. 8A and 8B, an alternative exemplary exo-graft 100′ has an axis of revolution 114, such that that a cross section of a lower part 110 of exo-graft 100′ does intersect with a cross section of an upper part 112 of exo-graft 100′ along a line parallel to a central longitudinal axis 114 of exo-graft 100′.

In an exemplary embodiment, exo-grafts 100, 100′ can be manufactured using conventional methods (e.g., molding, extrusion) or using additive manufacturing (e.g., 3D-printing). Polymers that are amenable to fabrication and have proven biocompatibility include: polyetheretherketone (PEEK), polypropylene (PP), polysulfone (PS), polyurethane (PU), ultra high molecular weight polyethylene (UHMWPE), polyvinylchloride (PVC), polyethersulfone (PES), polytetrafluoroethylene (PTFE), polyetherimide, (PEI), and polycarbonate (PC). In an exemplary embodiment, exo-grafts 100, 100′ can be constructed from a Dacron® mesh. Exo-grafts 100, 100′ can be an external add-on, to be deployed during the implantation of the MCS device. Alternatively, exografts 100, 100′ can be added by the surgeon just prior to implantation.

The length of exo-graft 100, 100′ is variable, and can extend between about 50 mm and about 120 mm, depending on the application and the size of the patient. The length can be dependent on the amount of spiral flow desired, limited only by the total length of the outflow graft 52 and localized anatomical constraints. The length of exo-graft 100, 100′ and its helical revolution are selectable and directly related to the amount of spiral content desired, and can be analytically optimized.

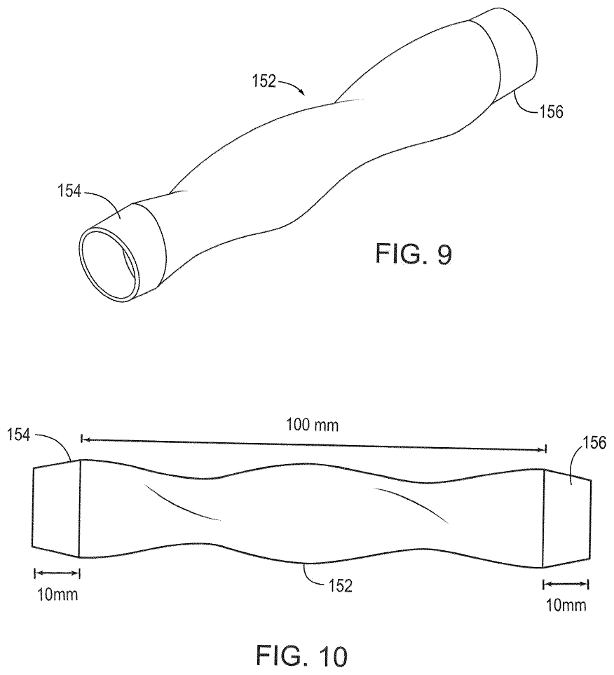

Referring now to FIGS. 9-15, different embodiments of exo-grafts are provided. FIGS. 9 and 10 show an exo-graft 152 having a working length of 100 mm, with tapered ends 154, 156 each having lengths of about 10 mm each. Exo-graft 152 has a rotational pitch of 100 mm, with one full revolution over its 100 mm length. In this exemplary embodiment, the rotation is in a clockwise direction.

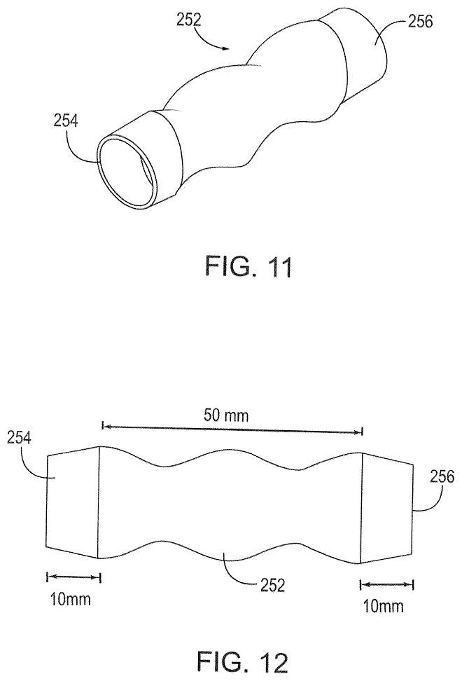

FIGS. 11 and 12 show an exo-graft 252 having a working length of 50 mm, with tapered ends 254, 256 each having lengths of about 10 mm each. Exo-graft 252 has a rotational pitch of 50 mm, with one full revolution over its 50 mm length. In this exemplary embodiment, the rotation is in a clockwise direction.

While exo-grafts 152, 252, exhibit a full revolution over their lengths, those skilled in the art will recognize that exo-grafts 152, 252 can exhibit as little as one half revolution over their lengths.

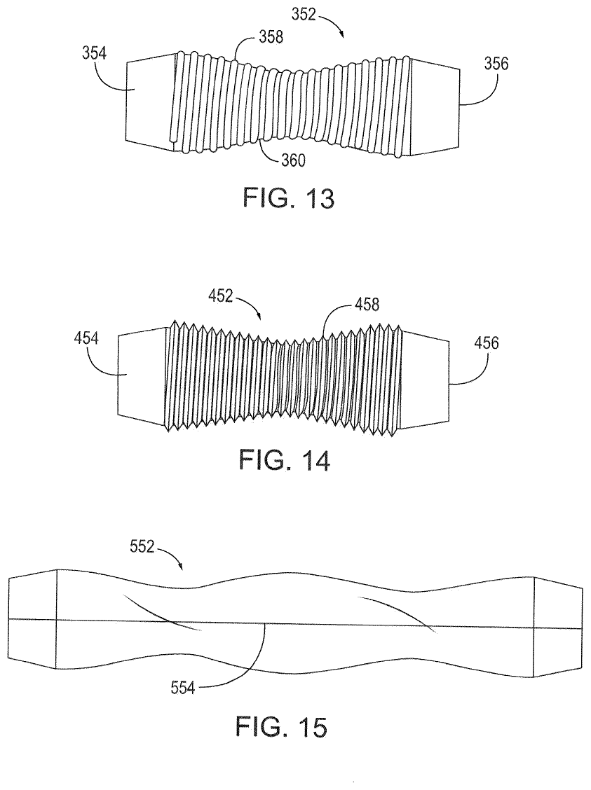

FIG. 13 shows an exo-graft 352 having tapered ends 354, 356 and a reinforcing spiral 358 wrapped around an exterior of exo-graft 352 between ends 354, 356. Spiral 358 is spaced such that a gap 360 is provided between adjacent wraps of spiral 358. Spiral 358 provides reinforcement to exo-graft 352 to act as a stiffener for graft 352.

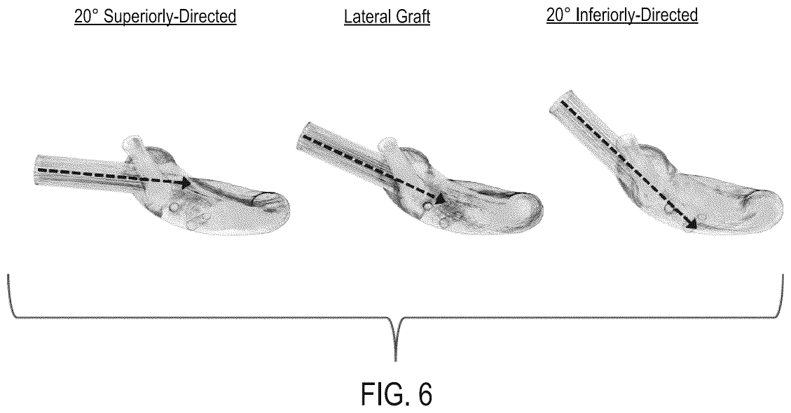

FIG. 14 shows an exo-graft 452 having tapered ends 454, 456 and an accordion body 458 extending between ends 454, 456. Accordion body 458 provides flexibility to exo-graft 452.

The inner diameter of the exo-grafts described above is designed to fully envelop the outflow graft 52 of the MCS 51. Typically, these exo-grafts are in the 10-16 mm inner diameter range.

It is entirely conceivable and may in fact be desirable to place the exo-graft 100, 100′ around large vessels (e.g., aorta, brachiocephalic, carotid, iliac, femoral) to modulate native flow and induce spiral flow. A longitudinal slit is made in the exo-graft and opened up so that the vessel can be inserted into the exo-graft through the slit. An exemplary exo-graft 552 with a longitudinal slit 554 is shown in FIG. 15. The material from which exo-graft 552 is constructed has a memory to close up the slit 554 after the native vessel is inserted into exo-graft 552. Alternatively, an attachment means (not shown) can be used to secure slit 554.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A mechanical circulatory support device spiral flow-inducing add-on comprising:
a pump configured for attachment to a heart;
an outflow graft having a first end in fluid communication with the pump and a second end configured for fluid communication with an aortic arch; and
a helically shaped exo-graft constrainment configured to wrap around at least part of the outflow graft to contour the outflow graft, thereby inducing spiral fluid flow inside the outflow graft.

2. The mechanical circulatory support device spiral flow-inducing add-on according to claim 1, wherein the pump has an output and wherein the helically shaped exo-graft constrainment is located at the output.

3. The mechanical circulatory support device spiral flow-inducing add-on according to claim 1, wherein the exo-graft is non-blood contacting.

4. The mechanical circulatory support device spiral flow-inducing add-on according to claim 1, wherein the exo-graft has a first end, a second end, and a longitudinal slit extending between the first end and the second end, such that the slit can be opened to envelope a native blood-carrying vessel.

5. The mechanical circulatory support device spiral flow-inducing add-on according to claim 1, wherein the exo-graft is configured to modulate the hemodynamics of a mechanical circulatory support outflow graft and to ameliorate device-mediated complications including at least one of jetting, vascular remodeling, thrombosis, stasis zones, and downstream endothelial dysfunction.

6. The mechanical circulatory support device spiral flow-inducing add-on according to claim 1, wherein the exo-graft has a generally arcuate/helical profile such that a cross section of a lower part of the exo-graft intersects with a cross section of an upper part of the exo-graft along a line parallel to a central longitudinal axis of the exo-graft.

7. A method of ameliorating device-mediated complications including at least one of jetting, vascular remodeling, thrombosis, and downstream endothelial dysfunction in a graft comprising the steps of:
providing the mechanical circulatory device according to claim 1; and
pumping blood from the pump and through the outflow graft, wherein the helically shaped constrainment induces spiral fluid flow inside the outflow graft.

8. A spiral flow-inducing exo-graft comprising a helical non-blood contacting constrainment adapted to wrap around a blood-carrying conduit.

9. The spiral flow-inducing exo-graft according to claim 8, wherein the exo-graft has a generally helical profile such that a cross section of a lower part of the exo-graft does not intersect with a cross section of an upper part of the exo-graft along a line parallel to a central longitudinal axis of the exo-graft.

10. The spiral flow-inducing exo-graft according to claim 8, wherein the exo-graft further comprises a first end, a second end, and a reinforcing spiral extending between the first end and the second end.

11. The spiral flow-inducing exo-graft according to claim 8, wherein the exo-graft further comprises a first end, a second end, and an accordion body extending between the first end and the second end.

12. The spiral flow-inducing exo-graft according to claim 8, wherein the exo-graft is configured to modulate the hemodynamics of a mechanical circulatory support outflow graft and to ameliorate device-mediated complications including at least one of jetting, vascular remodeling, thrombosis, stasis zones, and downstream endothelial dysfunction.

13. A graft assembly comprising:
an outflow graft having a first end, a second end, and a body extending between the first end and the second end, the outflow graft being configured to transmit blood between the first end and the second end; and
an exo-graft enveloping an exterior of the outflow graft, the exo-graft imposing a helically shaped constrainment on the outflow graft to contour the outflow graft, inducing spiral flow in the body.

14. The graft assembly according to claim 13, wherein the outflow graft is sized to extend between a pump and an aortic arch.

15. The graft assembly according to claim 13, wherein the exo-graft has a generally arcuate/helical profile such that a cross section of a lower part of the exo-graft intersects with a cross section of an upper part of the exo-graft along a line parallel to a central longitudinal axis of the graft.

16. The graft assembly according to claim 13, wherein the exo-graft has a first end, a second end, and a longitudinal slit extending between the first end and the second end, such that the slit can be opened to envelope a blood-carrying vessel.

17. The graft assembly according to claim 13, wherein the first end and the second end are tapered.

18. The graft assembly according to claim 13, wherein the exo-graft has rotational pitch of one full revolution over a length between the first end and the second end.

19. The graft assembly according to claim 13, wherein the exo-graft is non-blood contacting.

20. The graft assembly according to claim 19, wherein the exo-graft extends along a longitudinal central axis such that a cross section of the first end and a cross section of second end are both on the longitudinal central axis.
